(19) United States
(12) Patent Application Publication
(10) Pub. No.: US 2003/0014091 A1
(43) Pub. Date: Jan. 16, 2003

(54) Implantable wireless and battery-free communication system for diagnostics sensors

(76) Inventors: Jahangir S. Rastegar, Stony Brook, NY (US); Carios M. Pereira, Tannersville, PA (US)

Correspondence Address: Thomas Spinelli, 2 Sipala Ct, E. Northport, NY, 11731, US

(21) Appl. No.: 10/156,702

(22) Filed: May 28, 2002

Related U.S. Application Data
(60) Provisional application No. 60/293,621, filed on May 25, 2001.

(51) Int. Cl. A61N 1/08

(52) U.S. Cl. 607/61

(57) ABSTRACT

A device to be implanted in a body is provided. The device including: an internal power receiver for receiving a wireless transmission of power from a remote location; an energy storage device for storing the received power; and a processor at least partly powered by the energy storage device for carrying out an intended function. The device preferably further comprises a sensor operatively connected to the processor and energy storage device. The internal power receiver is preferably an antenna for receiving the external power transmission from an external transmitter.

### CROSS-REFERNCE TO RELATED APPLICATION

This application claims the benefit of earlier filed provisional patent application No. 60/293,621 filed May 25, 2001, entitled “Implantable Wireless And Battery-Free Communication System,” the contents of which are incorporated herein by its reference

### BACKGROUND OF THE INVENTION

1. Field of the Invention

The present invention relates generally to diagnostic sensors, and more particularly, to an implantable wireless and battery-free communication system for diagnostics sensors.

2. Prior Art

Implantable devices such as pace makers for sensing and performing certain tasks have been widely utilized in recent years. When one-way or two-way communication is required with the external world, wireless means of communication, particularly those using radio frequency have been widely used. Such bi-directional communication capability with implanted devices (systems) may be required or desired, for example if the device is used to monitor certain functions or signs via sensors or the like, or if command signals are needed to be transmitted to the implanted device for its proper operation.

The state of the art in wireless technology as applied to implantable devices is to provide the means to communicate the data to a common point, typically a system located external but close to the body. The current implantable devices require sources of power, usually batteries, for their operation. In certain devices, batteries have been used that can be recharged by an external source, usually by electromagnetic devices through the skin.

Internally placed batteries, however, have a number of shortcomings. Firstly, the batteries occupy a considerable amount of space as compared to that generally required for the electronics, sensors and the communication elements. As the result, in many cases, the size of the battery determines the overall size and sometimes even the shape of the implantable device. Secondly, all chemical batteries contain toxic materials that have to be protected from the body, thereby requiring various protective coatings and in some cases circuitry. Thirdly, if rechargeable, the battery requires a considerable amount of hardware for the recharging process and for safety. In addition, components of the recharging device must be implanted close to the skin to minimize the distance to the externally located component of the charging device and thereby maximize the rate of energy transfer and tissue exposure. The resulting power source and its accessories that are implanted in the body also occupies a considerable amount of space, and depending on the location of the sensory devices, may require relatively long wires to bring the power from the charging location to the sensor and its related hardware. The size issue obviously greatly limits the locations within the body at which such devices may be implanted. In addition, the larger the surface area of the implanted device, the higher will be the chances of complications such as infection, discomfort, body reaction, etc. The battery related space requirements have become an even greater factor limiting the development of implantable devices in recent years as advances in the micro-electronics and micro-electromechanical and related technologies have made it possible to manufacture extremely small sensors with integrated electronics circuitry and the required communications gear. The internally placed batteries have numerous other disadvantages such as heat generation, the possibility of malfunction and requirement to be replaced, limited life (shelf and operational), etc.

### SUMMARY OF THE INVENTION

Therefore it is an objective of the invention to provide an implantable wireless and battery-free communication system that can be used to operate implanted diagnostics sensors, medical instrumentation, medication delivery system, or the like (collectively referred to herein as a sensor).

Another objective of the present invention is to provide one-way or two-way communication between the implantable device and an external component that can be used for both data communication and for delivering power to the implantable device.

Another objective of the present invention is to provide the transfer of energy from outside the body to the implantable device by radio waves and the means to store and utilize the same.

Another objective of the present invention is to provide alternative (non-radio wave based) means of generating electrical energy within the body and the means to store and utilize the same.

Another objective of the present invention is to provide a co-located sensor, actuator and power source micro-integrated system that makes it possible to significantly reduce the size and volume of the device while significantly increasing its functionality.

Another objective of the present invention is to provide the methods and means of bi-directional communication capability between the implanted device and the external component of the system so that the power consumption can be minimized.

Still another objective of the present invention is to provide a one-way and two-way communication that can be encrypted and error checked.

Still yet another objective of the present invention is to provide the implanted device with the capability of performing self-diagnostics, sensor and the like calibration, adjustments, and the like. The implanted device may also be programmed to stay dormant while not being interrogated or not required to operate and be activated by an internal or external signal which may have been generated by an operator (directly or through other communication) or by an internal or external clock or initiated by an external or internal control signal.

Accordingly, a device to be implanted in a body is provided. The device comprising: an internal power receiving means for receiving a wireless transmission of power from a remote location; an energy storage device for storing the received power; and a processor at least partly powered by the energy storage device for carrying out an intended function.

Preferably, the device further comprises a sensor operatively connected to the processor and energy storage device.

Preferably, the internal power receiving means is an antenna for receiving the external power transmission from an external transmitter.

### BRIEF DESCRIPTION OF THE DRAWINGS

These and other features, aspects, and advantages of the apparatus and methods of the present invention will become better understood with regard to the following description, appended claims, and accompanying drawings where:

FIG. 1 illustrates a simplified schematic illustration of a preferred implementation of the present invention in which power is received by an external RF transmission.

FIG. 2 illustrates a more complicated schematic of a preferred implementation of the present invention.

### DETAILED DESCRIPTION OF THE PREFERRED EMBODIMENT

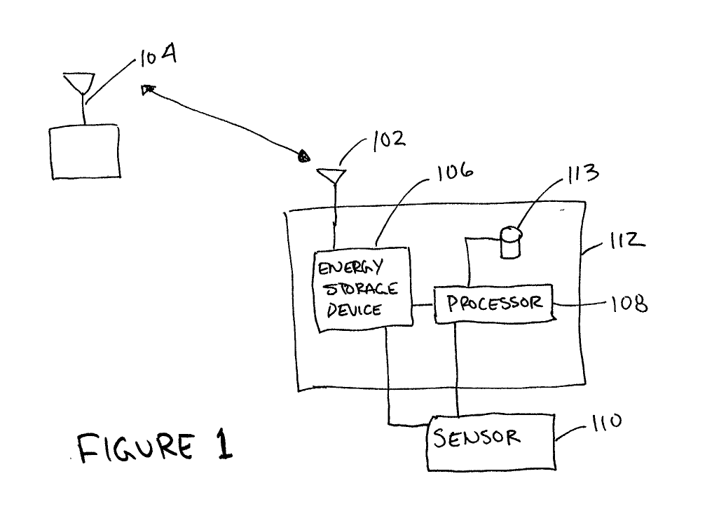

Referring now to FIG. 1, in one preferred embodiment of this invention, an implanted antenna 102 with a conformal design and capable of communicating on multiple frequencies are used to communicate to a second external antenna 104. The implantable conformal dual frequency antenna 102 is used to communicate to the second external antenna 104. The internal RF antenna 104 conforms to the geometry of the application. Advantages of using a single dual frequency RF antenna is to reduce space occupied by the implantable RF antenna 102, resulting in significant RF antenna size reduction.

The internal Radio Frequency (RF) antenna is considered to be conformal since it is preferred to conform to the geometry of the application in terms of the available space and shape/size of the implanted system. The main advantage of using a single dual frequency RF antenna is that its implanted RF antenna 102 occupies a relatively small space, thereby significantly reducing the overall size of the implanted component of the system. As will be discussed below, the implanted antenna 102 converts an RF signal from the external antenna 104 to energy and stores the energy in an energy storage device 106, such as a battery or capacitor. The energy storage device 106 is used to power a processor 108 and sensor 110. The implanted antenna 102, processor 108, and energy storage device 106 are shown as a single unit 112, which is implanted at a convenient area of the body. However, the sensor 110 may be integral with the unit 112.

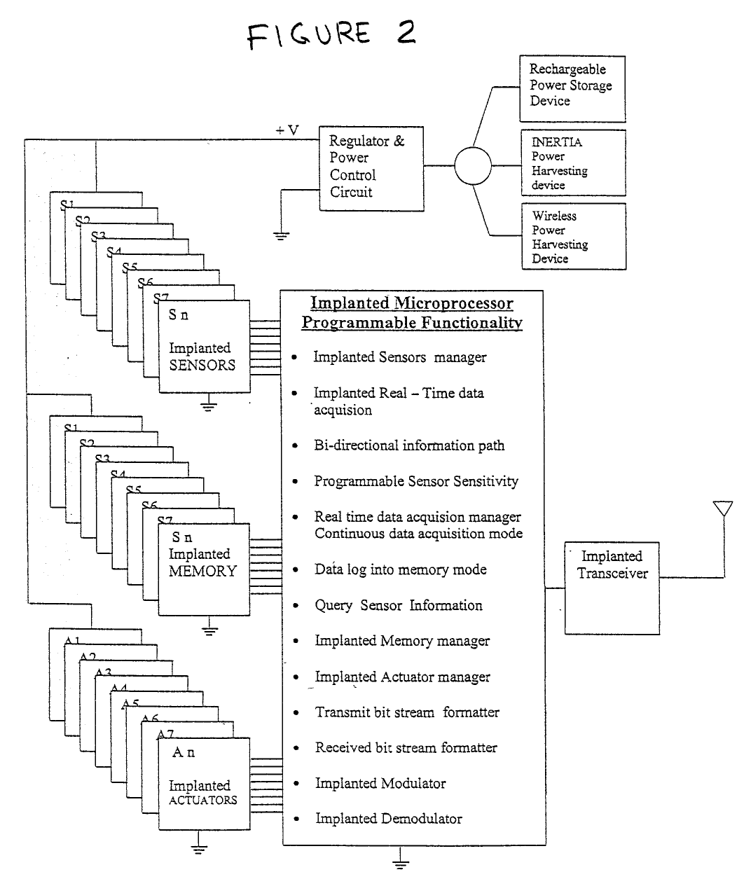

Referring now to FIG. 2, a micro power sensor and actuation system with wireless duplex communication capability makes it possible to design an integrated system with sensory, communication, actuation and an energy harvesting system that is capable of powering its own operation. This is accomplished by integrating the capabilities of two-way communication and by co-locating the functions of power harvesting, sensing, actuation and data processing. Adequate data processing and computational means can also be provided and similarly powered for proper operation of the system. Sufficient energy can be embedded into the RF carrier for the operation of such an implanted system. By properly designing RF antennas using techniques known to those with expertise in the art, very small antenna sizes can be achieved. By significantly reducing the size of the antennas, it becomes possible to construct the aforementioned implantable devices to fit in very small volumes on the order of a few cubic mm. Co-locating the power source, sensors and the actuators results in a micro integrated system with significantly advanced functionality.

This system is configured to perform functionality to include two-way communications and embedded into the RF carrier sufficient energy is transmitted to operate the sensor, actuators and communication system. The RF frequency transceiver uses special techniques to maintain the size of the RF antennas both at the receiving and the transmitting end to a minimum size. Reducing the antennas size to a minimum for these medical applications introduce a significant advancement in the applications of medical implantable devices because it makes it possible to adapt such implantable devices to a multitude of new applications.

In one embodiment of the present invention, energy is harvested from a Radio Frequency carrier that is being emitted from a transmitter 104 located outside the patient body. This method of powering implanted devices 112 has the added advantage of reducing the size and weight of the transmitting device. The Radio Frequency emitter source 104 can be placed anywhere outside the patient body and does not have to be carried by the patient. In fact, the emitter 104 need only be within a certain radial distance from the patient. The maximum radial distance is dependent on the power and frequency of the emitter, the antenna design and the amount of power required for the proper operation of the implanted device 112 (2-4 feet radius is readily achieved without requiring excessive power emission). This allows a person to have the freedom to have a normal lifestyle while his/her implanted device 112 is operating.

The implanted wireless device of the present invention has the unique ability to switch operational mode from RF device to internal storage device if the wireless mode of operation detects radio frequency interference. Advantages of this feature is to increase operational stability of the device and to prevent erroneous data or actions (sensor, actuator).

In another embodiment of the present invention, energy is generated by the motion-of a mechanical or electrical energy generating device due to the movement of the patient, a segment of his/her body, a muscle or the like. A number of such devices may be constructed, including:

1. A device operating on the pendulum principle. In such devices, a mass element is attached to a base structure by a spring element or by a joint (such as rotary, linear, spherical, etc., which are preferably living joints). The mass element may constitute part of the structure of the device. Spring elements may be linear, torsional, bending or any other type (even air or fluid type) or any of their combination most appropriate for each application. The base structure is in turn attached to the intended part of the patient body. As the patient body part moves in accelerating and decelerating modes horizontally, vertically or in combination, kinetic energy or potential energy or their combination are transferred to the mass element of the device. The movement of the patient may be voluntary or involuntary. The transferred energy is then harvested, preferably by transforming it into electrical energy utilizing well-known techniques and electrical power generation devices and elements.

2.In another embodiment, pure spring elements may be used directly between the aforementioned base elements that is attached to one point on the patient body and on its other end to another point on the patient body where the two points undergo relative motion during the voluntary or involuntary movement of the patient or his/or her organs. In such devices, the stored potential energy is then transformed into preferably electrical energy utilizing well-known techniques and electrical power generation devices and elements.

3. In another embodiment, the electrical power generator (based on magnets and coils or active materials such as piezoelectric materials) is directly attached to the patient body between two points that undergo relative motion as described above in a voluntary or involuntary mode. The electrical power generator can then generate electrical power directly.

4. In another embodiment, at least one end of either one of the above three embodiments are attached to a muscle. The power-generating device would then stimulate the muscle, thereby transferring mechanical energy to each device.

In general, and depending on the application and the health and physical condition and activity level of the patient, one or more of the aforementioned power generation devices may be utilized. The device may also be programmed to utilize one or another provided power generation device based on the power level requirements or status of the energy storage element or any other medical conditions that warrant the utilization of one electrical power generating device or the other. At times, it might even be appropriate to utilize more than one of the electrical power generating devices of the implanted device to generate electrical energy simultaneously. The power consumption budget is preferably closely integrated with the programmed functions and the modes of operation. The consumption of power can also be modified or adjusted remotely via the RF link, preferably by means of software modifications.

The electrical energy generated by any of the above electrical power generating devices is preferably stored in an inert storage device 106 within the implanted device 112. When rechargeable batteries with inert chemical composition become available, such batteries may also be used. The primary concern here is safety and the elimination of any accidental release of harmful chemicals in the patient body.

The implanted device 112 is capable of monitoring, communicating and actuating on a full time basis, however, to minimize power consumption, other modes of operation are available. In one mode of operation, the unit can be set (preferably remotely) to a monitoring mode during which the system (usually sensors) collect information at required time or event intervals. In this (monitoring) mode, the system operates primarily as a memory device and continues to log sensor information. In this mode of operation, it may not be necessary to establish full (duplex) communication with the implanted device. In this mode of operation, the implanted device can be queried at any time, at which time the duplex mode of communication is activated through the wireless link. In this mode of operation, whenever the implanted device or the outside part of the system detects a predetermined condition(s) (directly or after the examination of the data by a third party, etc.), the system can switch to a wireless full duplex mode to transmit an alarm signal, for the implanted device to receive proper instructions for proper action, or in short initiate a process that would lead to proper action(s).

In another mode of operation, the implantable device operates as a real-time device, during which mode the external diagnostic equipment would receive continuous information from the implanted device. Other functions, e.g., sensory functions or drug administration functions, etc., can also be continuously programmed or the existing programs be continuously modified or the parameters of the device or of the programs continuously set and reset during this mode of operation. For example, the sensors within the implanted device may be programmed to perform different types of measurements or have several sensors configured to perform certain measurements, etc. or their gain and frequency response to be changed. As such, the implanted device can be made to function as an adaptive and robust (intelligent) system that can adapt itself to the requirements of its environment and mission.

The operation of the implantable device can, therefore, be categorized into the following three major modes: (1) a real-time data acquisition mode with bi-directional information communications, (2) a second mode configured to sense and store information for extended periods of time, and (3) a reprogramming mode during which the unit shall receive instructions to reconfigure to the desired configuration and operational mode.

In the real-time data acquisition mode, the implanted device can send commands to the outside system or receive commands from the outside system for its various functions. During this mode, the embedded power storage device 106 has the opportunity to replenish its stored energy if RF carrier is used for power transmission purposes. In this mode, the implanted device has the highest rate of energy consumption.

The second mode of operation is designed to provide the longest autonomous mode of sensing, information storage and drug administration or the actuation of other types or elements that are provided in the implanted device. The energy consumed in this mode is relatively small. As the result, the operation of the implanted device can be sustained for very long periods of time. In this mode, the implanted device can be programmed to look for an alert condition and immediately switch to the real-time data acquisition mode. The commonly used safety features that ensure that the stored data is not lost can be readily programmed into the device. For example, the device can be programmed to perform self-checking operations such as check for low internally stored energy levels and dump the stored data to the external wireless device or save it on a protected internal memory device.

The third mode of operation is configured to provide the ability to perform the programming steps. In this mode, the implantable device enters a learning mode for the purposes of configuring memory, sensors, etc., and perform tasks such as sensor and actuator calibration and the like. In this mode, device architecture can be modified by means of software, which reconfigures the microchips that holds the instructions for the proper operation of the system, including all the embedded memory, sensors, actuators transceiver intercommunications protocols, etc.

The following features can be readily incorporated into the design of the aforementioned implantable device:

SWITCH MODE TO INTERNAL STORAGE IF THERE IS RF INTERFEERENCE: Such implanted wireless devices can be designed with the capability of switching their operational mode from RF device to internal storage device when the wireless mode of operation detects radio frequency interference. This feature greatly increases the operational stability of the implanted device and prevents erroneous reading of data (e.g., from sensors) or actions (e.g., by actuator).

INCORPORATIN OF ADVANCED DATA ENCRYPTION TECHNIQUES: By using advanced data encryption techniques, it can be ensured that the implanted device sends and receives the intended messages and responds to the intended command. Such advanced encryption techniques have shown to be highly effective for checking for errors in the transmitted information. Significant steps are taken to insure that the implantable device receives the intended message and responds to the intended command. This is part of an advanced encryption technique to check the transmitted information for errors. The implanted medical devices perform life/death types of functions, it is extremely important that the transmitted commands are interpreted and actuated precisely.

MONITORING FOR INTEGRITY OF INFORMATION RECEIVED BY IMPLANTED DEVICE: This feature is preferably implemented by writing remote commands to the memory 113 that is onboard the implanted device. A delay is also built in to allow for the data that is received and checked for accuracy. Such a feature (implemented as described or using a similar technique) ensures the integrity of the information received by the implanted device. These features can be implemented by writing remote commands to memory onboard the implanted device, followed by a built in delay to allow the data received to be checked for accuracy. These actions monitor the integrity of the information being transmitted.

IMPLANTABLE DEVICE SELF-MONITORING OF RADIO FREQUENCY SIGNAL STRENGTH. By employing techniques known to those skilled in the art, additional steps are taken to ensure that the radio frequency carrier does not introduce error into the information flow. One method consists of providing the implanted device with the means to detect radio frequency interference. When such interference is detected, the information flow is routed and stored in onboard memory, which is built into the implanted system.

Self-diagnostic capability can be readily incorporate into the implantable device using existing technology. In general, such capability is software driven. Special hardware may also be utilized, particularly for operations such as self-calibration of sensors and/or actuation devices. The self-diagnostic task may be initiated internally as predetermined time or events or may be communicated to the device from the outside part of the system.

The wireless implanted device is designed and packaged to operate in various extreme harsh environments, to include large temperature variations, shock loads, chemical corrosion, long-term usage and electronic interference. The implanted sensors and associated processor has the ability to detect radio frequency interference, if this is detected information flow is routed and stored on memory which is built into the implanted system.

The wireless implanted device is packaged or coated with currently available biocompatible material for safe and long term operation without corrosion within the body. Commonly used techniques are used to make the device resistant to shock load levels that are experienced within the body and to guard against electronic interference. The low power operation of the device also ensures that the device is maintained very close to the body temperature.

The implanted device may be equipped with the following, primarily software based, major adaptive (intelligent) capabilities:

1. The sensor suite may be reconfigured to various sensitivities and responses. This implies that each sensor may be programmed to respond to a preset level of stimuli and dynamic range to perform a specific measurement.

2. The memory bank(s) may be internally or externally partitioned to various memory block sizes to accommodate various lengths of the sensory data. The partitioning may be implemented via the wireless remote link. Such custom programmed memory allocation is particularly useful for storage of sensory data during the monitoring mode of the implantable device and allows the implanted system to continue to store information for very long periods of time.

3. The implantable device functionality can be made to be adaptable to each specific application by providing the capability to reconfigure the device using software. A combination of hardware and software may also be used to greatly increase the possible range of reconfigurabiliry.

Internal to the implantable device, the hardware is basically comprised of five main system blocks: (1) the power-harvesting module; (2) the microprocessor module; (3) the sensor module; (4) the memory module; and (5) the actuated elements module.

In a preferred implementation, energy is extracted from the RF carrier by unique method embedded in the implantable device. Power transfer is performed by totally remote methods. No contact with patient tissue. One advantage is to reduce the possibility of allergic reactions with the patient skin. Current methods to charge implantable devices require direct contact. Wireless methods to transmit and store power allows to power remote implantable devices from a vicinity location. The external transmitter may be located within a radius of approximately three feet. This method allows significant versatility to locate the power charging device. Reduction in weight is most significant on the external recharging device and also to a lesser degree on the implantable device. This significantly reduces the weight of current methods to charge remote medical devices

General methods to harvest and store energy for micro-power implantable devices. Storage devices in the implantable device are capable of harvesting energy from sources that contain potential energy. Energy is removed from an Radio Frequency carrier being emitted by a transmitter located outside the patient. This method of powering implantable microdevices is designed to reduce the size and weight of the transmitting device located outside the patient. The innovation consists of harvesting energy by an implantable device from a radio signal being emitted by an RF source located outside the patient's body. The Radio Frequency emitter source can be placed anywhere outside the patient's body and not necessarily carried by the patient. This allows a person to have the freedom to have a normal lifestyle while carring implantable devices. Such devices receive their power from an RF carrier, which radiates from a nearby source. The RF emitter can transmit energy while placed in any direction and as long as it is within a radial distance of the patient.

Energy to operate the implanted system can also be harvested from converting energy which exists in performing work to move a mass from point A to point B into electrical energy that can be stored for use by the implanted device. A typical example of such a system would be an inertial device or a piezo crystal device internally located on a person's muscle or diaphragm. The energy conversion device would convert mechanical motion into electrical energy that would be stored in an inert storage device within the implantable device. As the battery technology advances, batteries with inert chemical composition can also be used. A power harvesting and storage system is designed and embedded in the implantable device to store and deliver energy from multiple sources of energy harvesting devices. The energy collection is performed in parallel, energy is stored at a significant higher rate than the energy consumption to insure an ample power supply for the implantable devices. The power consumption budget is closely integrated with the programmed functions and the modes of operation. Furthermore, the consumption of power can be modified or adjusted remotely via the RF link, by means of software modifications.

IMPLANTED DEVICE CAN BE REPROGRAMMED REMOTELLY. This programs sensors functionality and response to their environment.

Advantages of being able to reprogram functionality are:

Use same sensor for various functions.

Reprogramming is done totally from the external environment, no need to remove the device and surgically implant the device every time the device needs maintenance.

Perform various functions with the same device. (e.g. Administer medicine, measure vital signs, internal search missions such as search and detect diseases in hard to find places.

Reconfigure the ability to harvest and store energy. This capability is different for various individuals because each person would have different levels of activity.

SELF-DIAGNOSTIC CAPABILITY. Implantable device has capability to store and report its internal status, such as sensor configuration functionality. This capability could offer the ability to internally monitor and report the rate at which the unit can harvest energy. This rate can be different for various individuals. Once it is know, it could be adjusted or customized for each individual.

Reconfigure implantable device to a real-time system or long term monitoring device. The implanted device is capable to monitor, communicate and actuate on a full time basis, however, to reduce the power consumption other modes of operation is implemented. It may not be necessary to establish full communications with the implantable device at all times, so the unit can be set remotely to a monitoring mode during which the sensor system can collect information at required time intervals. Furthermore the unit can be queried at any time, at which time another mode of operation can be selected through the wireless link. In the monitoring mode the system operates as a memory device continuing to log sensor information. If at any time an internal condition being monitored inside the patient generates an alarm or critical pre-programmed condition, the implantable device can automatically switch to a wireless full duplex mode and transmit the alarming condition to the external monitoring equipment.

The implantable sensor system operates as a real-time device, during which mode the external diagnostic equipment would receive continuous information from the implanted sensors. Other functions can also be programmed and modified during this mode. Internal sensors may be programmed to perform various functions, their gain and frequency response may be changed via the wireless link, and their memory may be interrogated to recover stored information. The implanted system is also designed to perform self-checking diagnostics to report internal status of the sensors functionality. The internal system consists of four major intelligent blocks:

The sensor suite may be reconfigured to various sensitivities, responses. This implies that each sensor is programmed to respond to a preset level of stimuli and dynamic range to perform a measurement. This level-can be changed for each sensor by external means via the wireless link. Modifications to the system performance is introduced by means of a software program to be downloaded to the onboard processor that describes the implantable device functionality. The memory banks can be externally partitioned to various memory block sizes to accommodate various lengths of sensor data. This feature is programmed via the wireless remote link and the custom programmed memory allocation is primarily useful for storage of sensor data during the implantable device monitoring mode, which allows the implantable system to continue to store information for very long periods of time.

The implantable device functionality is adaptable to the application by providing the capability to reconfigure the device using a balanced combination between hardware and software to reconfigure the hardware architecture. The functions of powering itself, sensing, communicating, actuating, built-in diagnostics, self-checking and self-calibration are major functions of the implantable device. Internally the hardware is comprised of five main system blocks: the power harvesting module, the microprocessor module, the sensor module, the memory module and the actuator module. The device architecture can be modified by means of software, which reconfigures formatting microchips that hold the instructions to manage, embedded memory, sensors, actuators and transceiver intercommunications protocols.

In summary, the implantable device operates in three major modes: (1) a real-time data acquisition mode with bi-directional information communications, (2) a second mode configured to sense and store information for extended periods of time, and (3) a reprogramming mode during which the unit shall receive instructions to select the appropriate configuration.

In the real-time data acquisition mode, it is possible to send commands to the implantable device and receive commands from the implantable device. In this mode it is also be able to send commands to perform internal actuation functionality. During this mode the embedded power storage device also has the opportunity to replenish the stored energy resources. The energy can be collected from the Radio Frequency carrier and from the embedded inertial devices. This mode also has the most energy consumption with the most dynamic functionality of the device.

The second mode of operation is programmed to provide the longest autonomous mode of sensing and information storage. The energy consumed in this mode is very little and therefore operation could be sustained for very long periods-of time. In this mode the implantable device could also be programmed to look for an alert condition and immediately switch to the real-time data. acquisition system. Safety features to insure that the data stored is not lost can be programmed, for example the device can self check itself and look for a low internal stored energy condition. If this occurs the implantable device dumps the information to an external wireless device.

The third mode of operation is configured to provide the ability to perform the programming steps. In this mode the implantable device enters a learning mode for the purposes of configuring memory and sensors, calibrate sensors and the actuators.

While there has been shown and described what is considered to be preferred embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

What is claimed is:

1. A device to be implanted in a body, the device comprising:
an internal power receiving means for receiving a wireless transmission of power from a remote location;
an energy storage device for storing the received power; and
a processor at least partly powered by the energy storage device for carrying out an intended function.

2. The device of claim 1, further comprising a sensor operatively connected to the processor and energy storage device.

3. The device of claim 1, wherein the internal power receiving means is an antenna for receiving the external power transmission from an external transmitter.
